(19) United States
(12) Patent Application Publication
(10) Pub. No.: US 2026/0328693 A1
(43) Pub. Date: Oct. 22, 2026

(54) METHOD OF RAMAN SPECTROSCOPY

(71) Applicant: Endress+Hauser Optical Analysis, Inc., Ann Arbor, MI (US)

(72) Inventors: Randy Benedict, Ann Arbor, MI (US); Karen Esmonde-White, Ann Arbor, MI (US); Mary Lewis, Ann Arbor, MI (US)

(21) Appl. No.: 19/305,437

(22) Filed: Aug. 20, 2025

Related U.S. Application Data
(63) Continuation-in-part of application No. 18/500,379, filed on Nov. 2, 2023, now Pat. No. 12,416,576.

(51) Int. Cl. G01N 21/65; G01N 21/84; G01N 33/03

(52) U.S. Cl. CPC G01N 21/65; G01N 21/84; G01N 33/03; G01N 2021/8466

(57) ABSTRACT

A method includes, with a Raman spectrometer: determining a measured spectrum; preprocessing the measured spectrum; determining a second derivative of a difference spectrum function corresponding to a difference between the measured spectrum and a product of a scaling coefficient and a reference spectrum with contributions of interfering influences; determining a coefficient value of the scaling coefficient minimizing an error function including a term corresponding to an error of the difference spectrum function due to peaks included in the reference spectrum, the term including a sum of areas enclosed underneath the second derivative in all spectral regions in which the second derivative is positive, and/or a sum of areas enclosed underneath the second derivative in all spectral regions in which the second derivative is negative; and determining a Raman difference spectrum corresponding to the difference between the measured spectrum and a product of the coefficient value and the reference spectrum.

### CROSS-REFERENCE TO RELATED APPLICATION

The present application is a continuation-in-part of U.S. patent application Ser. No. 18/500,379, filed Nov. 2, 2023, the entire content of which is incorporated herein by reference.

### TECHNICAL FIELD

The present disclosure relates to a method of Raman spectroscopy, in particular, a method for determining a measurement result of a measurand of a medium.

### BACKGROUND

Raman spectroscopy is a technique used to measure the wavelength and intensity of inelastically scattered radiation (e.g., light) from a sample, thereby revealing a chemical and structural composition of the sample. Raman spectroscopy is based on the principle that monochromatic excitation light will be reflected, absorbed, or scattered as a function of the particular molecule (e.g., protein, peptide, carbohydrate, cytokine, salt, etc.) that receives the incident radiation. Most of the energy is scattered at the same wavelength of the excitation light, referred to as elastic or Rayleigh scattering. A much, much smaller amount (e.g., ~0.001%) is scattered at different wavelengths (i.e., shifted), called inelastic or Raman scattering, the wavelengths of which are dependent on the molecular composition of the region sampled. In Raman spectroscopic analysis (interchangeably, Raman analysis and Raman spectroscopy), these wavelength shifts are captured in Raman spectra comprising the Raman scattered light (i.e., the Raman signal), which are analyzed to determine sample properties, including both chemical and physical properties.

Raman spectrometers are currently employed in a large variety of different applications, including industrial applications and laboratory applications to determine and provide measurement results of various measurands of a medium. As an example, Raman spectrometers are, e.g., employed to determine concentrations of components included in the medium, a pH-value of the medium, a melt index of the medium and/or a cell motility of the medium.

Raman spectrometers commonly include a light source transmitting monochromatic excitation light to a sample of the medium and a spectrometric unit receiving Raman scattered light emanating from the sample and determining and providing measured spectra corresponding to intensity spectra of the Raman scattered light. The measured spectra are, e.g., provided to an evaluation unit determining and providing measurement results of the respective measurand based on a previously determined model for determining the measurement results based on spectral intensities of the measured spectra.

Models used in Raman spectroscopy to determine measurement results of measurands can, for example, be determined based on measured spectra of samples exhibiting known values of the respective measurand and a detailed mathematical analysis of the sample spectra.

One of the problems associated to Raman spectroscopic measurements is, however, that at least some of the information on the measurand(s) contained in measured spectra of a medium may be obscured by contributions of interfering influences included in the measured spectra. As an example, measurements of a concentration of a target contained in a medium including multiple components may be impaired by contributions of an interfering analyte to the spectral intensity of the measured spectra in spectral regions of interest with respect to the determination of the concentration of the target analyte. With respect to media including a solute dissolved in a solvent, measurements of measurands of the solute may be impaired by contributions of the solvent to the spectral intensities of the measured spectra.

As another example, spectral information for determining a measurand may be obscured by contributions of light contaminations included in the light received by the spectrometric unit of the Raman spectrometer. Depending on the Raman spectroscopic instrumentation used and/or the application where it is employed, light contaminations, e.g., include fluorescent light emitted by fluorescent materials located along an optical path extending from the light source to the spectrometric unit that are illuminated by the excitation light.

In many applications, contributions of interfering influences included in the measured spectra make accurately determining the measurand(s) difficult or even impossible. Accordingly, there is a need to eliminate the contributions of interfering influences included in measured spectra.

One approach to achieve this goal is to determine Raman difference spectra by subtracting a reference spectrum of the contributions of the interfering influences from the measured spectra. As an example, a reference spectrum of contributions of an interfering analyte contained in a medium may be subtracted from measured spectra of the medium. To eliminate the contributions of interfering influences, the reference spectra have to be scaled according to a magnitude of the contributions of the interfering influences included in the measured spectra in a manner accounting for the intensity scale of the Raman spectrometer providing the measured spectra. Scaling is, e.g., performed by determining the spectral intensity values of the Raman difference spectra as differences between the spectral intensity values of the measured spectra and the product of a scaling coefficient and the spectral intensity value of the reference spectrum at the respective spectral line.

Determining the coefficient value of the scaling coefficient corresponding to the magnitude of the contributions of the interfering influences included in the measured spectra may, however, be a difficult task, in particular, in applications where the concentration of an interfering analyte is unknown or variable and/or where an intensity of light contaminations may vary. Under-subtracting by applying a coefficient value that is too small will result in Raman difference spectra in which spectral information for determining the measurand(s) may still be obscured by remaining contributions of the interfering influences. Over-subtracting by applying a coefficient value that is too large may alter or even remove spectral information that could otherwise be used to determine the measurand(s).

Whereas experimental and/or manual determinations of the coefficient value may be tedious and error-prone some progress has been made with respect to automating spectral subtractions.

The article “Second-Derivative Variance Minimization Method for Automated Spectral Subtraction” by Yvette L. Loethen, et al., published in 2004 in Applied Spectroscopy, Volume 58, Number 3, describes a second derivative minimization (SDVM) procedure for automatically extracting spectra of a dilute component included in a medium from measured spectra of the medium that are dominated by a major component, e.g., a solvent. According to this method, the scaling coefficient value for determining Raman difference spectra by subtracting a correspondingly scaled reference spectrum of the solvent from the measured spectra is determined such that it minimizes an error function given by the variance of a second order derivative of the smoothened Raman difference spectra.

As outlined in the article, this method provides the advantage of being less vulnerable to noise and fluorescence interferences than other methods. The use of this method is, however, limited to applications in which the variance of the second order derivatives of the smoothened Raman difference spectra exhibit a minimum at the optimum value of the scaling coefficient, which minimum is pronounced enough to enable a sufficiently accurate determination of the scaling coefficient value minimizing the variance.

This aspect makes the method unsuitable for applications in which the variance is a shallow function of the scaling coefficient. The latter is, e.g., the case in applications where the solute spectra and the solvent spectra are highly correlated and/or include overlapping spectral peaks. The shallower the error function is, the larger is the error associated to the determination of the scaling coefficient value minimizing the function. Depending on the degree of correlation and/or spectral overlap, the error function may be so shallow that the error associated to the determination of the minimum exceeds a maximum permissible tolerance.

The article “Asymmetric least-square baseline algorithm with peak screening for automatic processing of the Raman spectra” by Vitaly I. Korepanov, published in the Journal of Raman Spectroscopy, 2020; 1-5 (https://doi.org/10.1002/jrs.5952), discloses a method of determining a smooth baseline representing the contributions of interfering influences included in measured Raman spectra by minimizing an error function given by a sum of two terms. The first term is an asymmetric weighted sum of the squares of the differences between the spectral intensity values of the measured spectrum and the baseline, in which weighing factors applied to differences due to spectral intensity values of the measured spectrum exceeding the corresponding spectral intensity value of the baseline are substantially lower than weighing factors applied to differences due to spectral intensity values of the measured spectrum subsiding the corresponding spectral intensity value of the baseline. The second term is given by the product of a constant penalty factor and the sum of the squares of the second derivative of the baseline at the individual spectral lines.

As recognized in this article, in peak regions of the measured spectrum the differences between the spectral intensity values of the measured spectrum and the baseline may become large enough to cause undesired inflections of the estimated baseline. According to the article, this problem can be solved by performing an iterative process of determining the weighing factors in a manner accounting for the peak regions.

Asymmetric least square algorithms (ALS), like the algorithms described in the article cited above, can also be used to determine coefficient values of scaling coefficients for determining Raman difference spectra by subtracting a scaled reference spectrum of contributions of interfering influences from the measured spectra. In this context, they provide good results with respect to eliminating contributions of interfering influences, such as broad band luminescence light, exhibiting fairly smooth intensity profiles.

A disadvantage of corresponding ALS algorithms is, however, that they penalize the second derivative of the reference spectra. This fundamentally increases the penalty applied to any peak region the reference spectrum may exhibit. As a result, scaling coefficient values determined based on ALS algorithms for reference spectra exhibiting distinct peaks tend to be too small. This leads to under-subtraction resulting in Raman difference spectra, where valuable spectral information may still be obscured by contributions of interfering influences remaining in the Raman difference spectra.

Accordingly, there remains a need for further contributions in this area of technology. As an example, there is a need for a method that is more capable of eliminating contributions of interfering influences included in measured spectra in applications, where the contributions exhibit a peaky reference spectrum and/or a reference spectrum including spectral features that are correlated to and/or at least partially overlap with other spectral features included in the measured spectra.

Evaluating mediums or components in a medium where there are such interfering influences is a common step in industrial processes for quality control and/or regulatory compliance. Such analyses may be used, for example, to determine the concentration or presence of a particular chemical species in the medium. For example, alkane solvents, such as pentane and hexane, are commonly used in the processing of foodstuffs, including canola oil. Similarly, naturally occurring alkanes, such as those found in the cuticular wax of legumes, can also be present in byproducts following oil extraction, such as solid bean waste or meal.

Residual concentrations of such alkanes may ultimately affect the quality, stability, or suitability of the final product for specific applications. Further, regulatory agencies have established stringent limits on permissible alkane residue levels in food products, for example, in the parts-per-million (ppm) range. Therefore, monitoring the concentration of various analytes during processing is a critical task requiring sensitive detection methods. For example, analyses in the food and beverage industry may require detecting the presence of alkanes in samples at as low as 2500 ppm.

Conventional analytical techniques for detecting and quantifying alkane concentrations at such levels include gas chromatography, high-performance liquid chromatography, and flame ionization detection. While these methods offer high sensitivity and specificity, they are inherently offline processes. As such, they require extensive sample preparation, trained personnel, and significant time investment, making them unappealing for real-time or high-throughput industrial environments, frequently leading to bottlenecks in the production process.

Inline monitoring approaches are therefore highly desirable, as they enable real-time analysis without interrupting production workflows. Raman spectroscopy presents a promising solution for inline chemical analysis due to its non-destructive nature and ability to provide information with atomic-level resolution. However, the application of Raman spectroscopy in inline monitoring of complex matrices remains limited. A primary challenge lies in the spectral complexity of the sample matrix, where strong Raman signals, for example, from the oil components, can obscure or overlap with the spectral bands of target analytes, for example, alkanes, complicating data interpretation and reducing detection accuracy. Accordingly, there remains a need for improved methods and systems for Raman spectroscopy that are capable of accurately evaluating an analyte from spectra with many interfering influences.

Raman difference spectroscopy, wherein reference spectra representing known interfering influences are subtracted from the measured spectra, is a viable technique for coping with such interferences. Such approaches can significantly enhance the clarity and interpretability of spectral data by isolating features directly related to the analyte of interest. However, the development of such models that are capable of reliably correlating these spectral features with a target property at a low detection level remains a complex and challenging task. For example, factors like scattering effects and baseline shifts can obfuscate spectral signals, complicating the construction of analytic models. Therefore, there is a continued need for development in this area of technology.

### SUMMARY

The present disclosure includes a method of Raman spectroscopy for performing Raman spectroscopic measurements of at least one measurand of a medium in at least one application, wherein measured spectra of the medium include contributions of interfering influences that at least partially obscure spectra features associated to the at least one measurand, the method comprising, in at least one application:
with a Raman spectrometer, determining a measured spectrum of the medium;
providing a reference spectrum of the contributions of the interfering influences included in a respective measured spectrum, wherein a mean of the reference spectrum is equal to zero and a standard deviation of the reference spectrum is equal to one;
determining an adjusted spectrum from the measured spectrum of the medium, wherein a mean of the adjusted spectrum is equal to zero, and a standard deviation of the adjusted spectrum is equal to one;
determining a difference spectrum function corresponding to a difference between a product of a scaling coefficient and the reference spectrum;
determining a second derivative of the difference spectrum function with respect to a spectral line;
determining a coefficient value of the scaling coefficient corresponding to a magnitude of the contributions of the interfering influences included in the respective adjusted spectrum such that the coefficient value minimizes an error function representing an error of the difference spectrum function as a function of the scaling coefficient, wherein the error function includes a first term corresponding to an error exhibited by the difference spectrum function due to peaks included in the reference spectrum, the first term including at least one of:
a first sum of areas enclosed underneath the second derivative of the difference spectrum function in all spectral regions in which the second derivative of the difference spectrum function is positive; and
a second sum of areas enclosed underneath the second derivative of the difference spectrum function in all spectral regions in which the second derivative of the difference spectrum function is negative; and
determining and providing a Raman difference spectrum corresponding to the difference between the adjusted spectrum and a product of the coefficient value and the reference spectrum.

In at least one embodiment, the method further comprises determining a measurement result of the at least one measurand of the medium based on the Raman difference spectrum or based on more than one Raman difference spectrum.

In at least one embodiment, the method further comprises, for the at least one measurand, determining a model for determining measurement results of each respective measurand based on the Raman difference spectrum determined from adjusted spectra of samples of media exhibiting known values of the respective measurand.

In at least one embodiment of the method, the model for determining measurement results of the respective measurand comprises a partial least squares regression, wherein the partial least squares regression is fitted from Raman difference spectra of samples of media exhibiting known values of the respective measurand.

In at least one embodiment of the method the respective measurand comprises the concentration of an analyte in the medium.

In at least one embodiment of the method, the medium comprises oil, and the analyte is an alkane in the oil.

In at least one embodiment of the method, the concentration of the alkane in the medium is detected at a detection limit of less than 1,000 ppm.

In at least one embodiment of the method, the medium comprises bean meal byproduct from an initial oil extraction, and the analyte is an alkane in the bean meal byproduct.

In at least one embodiment of the method, the step of determining the adjusted measuring spectrum and adjusted reference spectrum comprises trimming the measured spectrum and/or the reference spectrum such that at least one portion of the measured spectrum and/or the reference spectrum is excluded from subsequent method steps.

In at least one embodiment of the method, the method further comprises:
with a smoothing filter, a second or higher order smoothing filter, a Savitzky-Golay filter, a second or higher order Savitzky-Golay filter, a rolling ball filter, or a wavelet decomposition filter, filtering the adjusted spectrum and the reference spectrum; and
determining the difference spectrum function and/or the second derivative of the difference spectrum function based on the filtered adjusted spectrum and the filtered reference spectrum.

The present disclosure also includes a Raman spectroscopic measurement system configured to perform the method according to claim 1, the Raman spectroscopic measurement system comprising:
a Raman spectrometer configured to determine and provide measured spectra of a medium; and
a difference spectra determination unit configured to determine and provide Raman difference spectra based on the measured spectra and a reference spectrum of contributions of interfering influences included in the measured spectra provided to the difference spectra determination unit.

The present disclosure also includes a Raman spectroscopic measurement system, comprising:
a Raman spectrometer, comprising:
a monochromatic light source configured to transmit excitation light having a predetermined excitation wavelength a measurement region configured to accommodate a sample of a medium as to illuminate the sample; and
a spectrometric unit configured to receive Raman scattered light emanating from the illuminated sample and to provide measured spectra, including intensity spectra of the Raman scattered light, wherein the spectrometric unit includes a disperser, a detector, and a signal processor configured to determine and to provide spectral intensity values of the measured spectra based on signals from the detector; and
a difference spectra determination unit configured to determine Raman difference spectra based on the measured spectra
wherein the signal processor of the spectrometric unit and the difference spectra determination unit are cooperatively configured to perform the method according to claim 1.

In at least one embodiment, the Raman spectroscopic measurement system further comprises a signal processing unit configured to determine and provide a measurement result of the at least one measurand of the medium based on the Raman difference spectrum provided by the difference spectra determination unit and on a previously determined model for determining the measurement result based on spectral intensity values of the Raman difference spectra.

In at least one embodiment of the Raman spectroscopic measurement system, the previously determined model is a partial least squares regression model fitted from Raman difference spectra of samples of media exhibiting known values of the measurand.

The present disclosure also includes a non-transitory computer-readable medium storing instructions that, when executed by one or more programmable processors, cause the one or more programmable processors to perform operations for performing a method of Raman spectroscopy comprising determining and providing Raman difference spectra for performing Raman spectroscopic measurements of at least one measurand of a medium in at least one application, wherein measured spectra of the medium include contributions of interfering influences that at least partially obscure spectra features associated to the at least one measurand, based on a measured spectrum of the medium determined by a Raman spectrometer and a reference spectrum of the contributions of the interfering influences included in a respective measured spectrum, the operations comprising:
determining a second derivative with respect to a spectral line of a difference spectrum function corresponding to a difference between the respective measured spectrum and the product of a scaling coefficient and the reference spectrum;
determining a coefficient value of the scaling coefficient corresponding to a magnitude of the contributions of the interfering influences included in the respective measured spectrum such that the coefficient value minimizes an error function representing an error of the difference spectrum function as a function of the scaling coefficient, wherein the error function includes a first term corresponding to an error exhibited by the difference spectrum function due to peaks included in the reference spectrum, the first term including at least one of:
a first sum of areas enclosed underneath the second derivative of the difference spectrum function in all spectral regions in which the second derivative of the difference spectrum function is positive; and
a second sum of areas enclosed underneath the second derivative of the difference spectrum function in all spectral regions in which the second derivative of the difference spectrum function is negative;
for at least one measured spectrum determined by the Raman spectrometer, determining a Raman difference spectrum corresponding to the difference between the respective measured spectrum and the product of the coefficient value and the reference spectrum; and
applying a previously determined model to the Raman difference spectrum to determine a measurement result of the medium.

In at least one embodiment of the non-transitory computer-readable medium, the previously determined model comprises a partial least squares regression.

### BRIEF DESCRIPTION OF THE DRAWINGS

The described embodiments and other features, advantages and disclosures contained herein, and the manner of attaining them, will become apparent and the present disclosure will be better understood by reference to the following description of various embodiments of the present disclosure taken in junction with the accompanying drawings, wherein:

FIG. 1 shows a flow chart of a method of Raman spectroscopy according to the present disclosure;

FIG. 2 shows a Raman spectrometric measurement system according to the present disclosure;

FIG. 3 shows an exemplary intensity profile of a measured spectrum;

FIG. 4 shows an exemplary intensity profile of a reference spectrum exhibiting a constant baseline;

FIG. 5 shows an exemplary intensity profile of a reference spectrum exhibiting a smooth baseline;

FIG. 6 shows a difference spectrum function determined by under-subtraction;

FIG. 7 shows a second derivative of the difference spectrum function shown in FIG. 6;

FIG. 8 shows a difference spectrum function determined by applying a coefficient value of a scaling coefficient corresponding to a magnitude of interfering influences included in the measured spectrum shown in FIG. 3;

FIG. 9 shows a second derivative of the difference spectrum function shown in FIG. 8;

FIG. 10 shows a difference spectrum function determined by over-subtraction;

FIG. 11 shows a second derivative of the difference spectrum function shown in FIG. 10;

FIG. 12 shows an error function; and

FIG. 13 shows a flow chart of a method of Raman spectroscopy according to the present disclosure.

In the figures, the same features are identified by the same reference signs.

### DETAILED DESCRIPTION

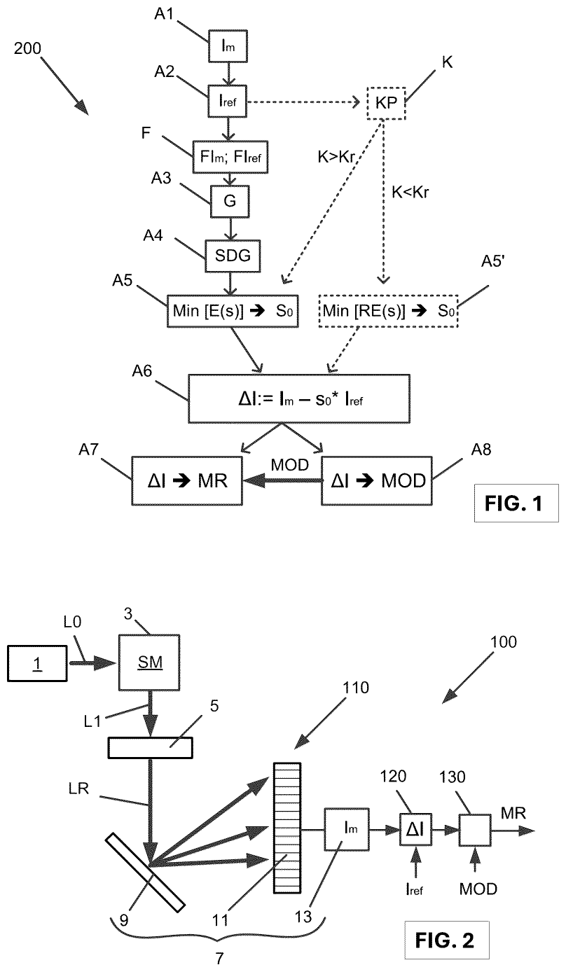

The present disclosure includes a method of Raman spectroscopy comprising determining and providing Raman difference spectra ΔI for performing Raman spectroscopic measurements of at least one measurand of a medium in at least one application, wherein measured spectra Iₘ of the medium include contributions of interfering influences, e.g., contributions that at least partially obscure spectra features associated to the measurand(s). A flow chart of the method 200 according to the present disclosure employed in each application is shown in FIG. 1.

A Raman spectroscopic measurement system 100 for performing the method 200 is shown in FIG. 2. The Raman spectroscopic measurement system 100 includes a Raman spectrometer 110 configured for determining and providing measured spectra Iₘ of the medium and a difference spectra determination unit 120 configured for determining and providing corresponding Raman difference spectra ΔI based on the measured spectra Iₘ, cooperatively configured for performing the method 200 shown in FIG. 1.

The exemplary Raman spectrometer 110 shown in FIG. 2 includes a monochromatic light source 1, e.g., a laser, configured to transmit excitation light L0 having a predetermined excitation wavelength λ₀ to a measurement region 3 configured to accommodate a sample SM of the medium.

In certain embodiments, the excitation wavelength λ₂ is, e.g., a wavelength in the visual or near infrared wavelengths range, e.g., a wavelength of 785 nm or of 532 nm. In certain embodiments, the Raman spectrometer 110, e.g., includes a filter 5, e.g., a notch-filter, configured to filter out Raman scattered light LR included in light LI emanating from the measurement region 3.

The Raman spectrometer 110 further includes a spectrometric unit 7 configured to receive Raman scattered light LR emanating from the illuminated sample SM. The spectrometric unit 7 is configured to determine and to provide the measured spectra Iₘ. The measured spectra Iₘ are, e.g., provided as intensity spectra of the Raman scattered light LR in a predetermined spectral range Δλ, e.g., a wavelength range or a wavenumber range of wavenumber shifts of Raman scattered light induced by excitation light L0 having the excitation wavelength λ₀.

In certain embodiments, the spectrometric unit 7, e.g., includes a disperser 9, e.g., a diffractive or holographic grating, dispersing the incident measurement light LR, a detector 11 receiving the dispersed Raman scattered light LR, and a signal processor 13, e.g., a microprocessor, connected to the detector 11. The detector 11 is configured to determine and to provide detector signals corresponding to spectral intensities of the incident dispersed measurement light LR. The signal processor 13 is configured to determine and to provide spectral intensity values I_{m,i} of the measured spectra Iₘ based on the detector signals.

The difference spectra determination unit 120 is, e.g., a computer, a microprocessor or another type of programmable calculating device including a memory, e.g., non-transitory computer readable medium, and configured to determine the Raman difference spectra ΔI based on the measured spectra Iₘ by executing a computer program, e.g., instructions executable by a processor, installed on the difference spectra determination unit 120.

In each application, the method 200 includes a method step A1 of, with the Raman spectrometer 110, determining a measured spectrum Iₘ of the medium, and a method step A2 of providing a reference spectrum I_{ref} of the contributions of the interfering influences included in the measured spectrum Iₘ.

Depending on the application, the interfering influences contributing to the measured spectra Iₘ, e.g., include light contaminations, e.g., luminescence or fluorescence light, exhibiting at least one intensity peak, and/or influences of an interfering analyte, of a composition of interfering analytes and/or of a solvent included in the medium. In addition or as an alternative, the interfering influences, e.g., include background influences, e.g., broad band luminescence or fluorescence light, exhibiting an at least approximately smooth intensity profile.

As used in the present disclosure, the term “peak” refers to a localized maximum of spectrum or function, which may have multiple localized maxima, and the term “valley” refers to a localized minimum of spectrum or function, which may have multiple localized minima. Accordingly, “peakiness” refers to the relative prevalence of localized maxima in a spectrum or function.

In certain embodiments, providing the reference spectrum I_{ref}, e.g., includes with a Raman spectrometric instrument determining and providing at least one intensity spectrum of the interfering influences and determining and providing the reference spectrum I_{ref} based on these intensity spectra.

As an example, in certain embodiments, determining the reference spectrum I_{ref}, e.g., includes determining at least one intensity spectrum of the interfering analyte(s) and/or the solvent included in the medium. The determination of each intensity spectrum is, e.g., performed by a highly accurate spectroscopic measurement instrument and/or under laboratory conditions.

The reference spectrum I_{ref} is, e.g., determined and/or provided as a normalized spectrum representing the spectral intensity distribution of the reference spectrum I_{ref} on a normalized intensity scale, e.g., by determining spectral intensity values of the reference spectrum I_{ref} as relative intensity values with respect to the maximum intensity value included in the reference spectrum I_{ref}.

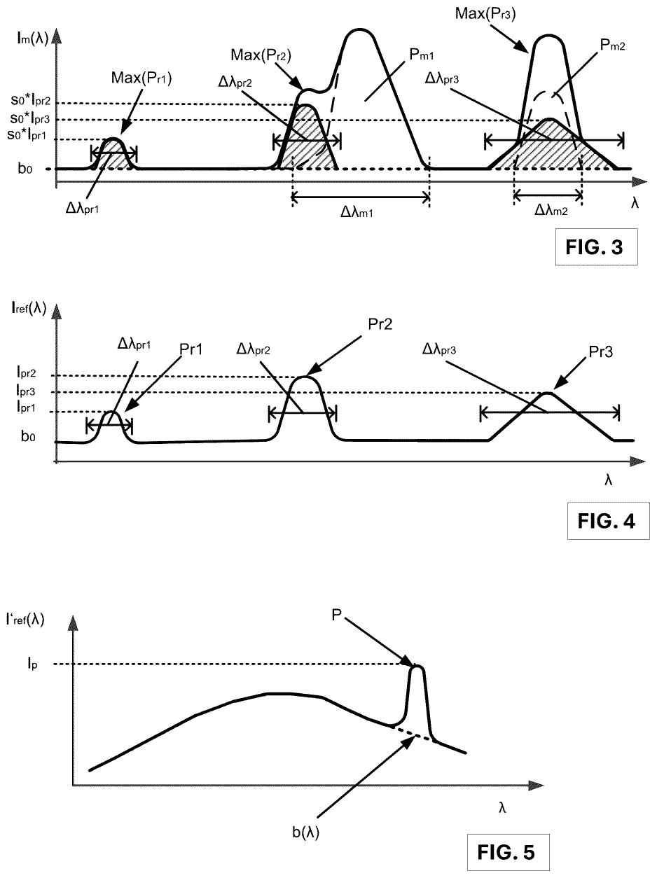

An exemplary intensity profile Iₘ(λ) of the measured spectrum Iₘ determined in method step A1 is shown in FIG. 3. FIG. 4 shows an exemplary intensity profile I_{ref}(λ) of the reference spectrum I_{ref} of the contributions of the interfering influences included in the measured spectrum Iₘ shown in FIG. 3.

In the example shown, the reference spectrum I_{ref} is a peaky spectrum exhibiting multiple distinct peaks Pr1, Pr2, Pr3 superimposed on a constant baseline b(λ):=b₀. Even though the method 200 disclosed herein is subsequently described based on the exemplary intensity profiles Iₘ(λ), I_{ref}(λ) shown in FIGS. 3 and 4, the method 200 can be performed in the same way in applications in which the reference spectrum I′_{ref} exhibits a smooth baseline b(λ), e.g., a baseline b(λ) caused by background influences, e.g., broad band luminescent or fluorescent light, exhibiting a smooth intensity profile, e.g., a profile including gradually curved sections. An example of an intensity profile I′_{ref}(λ) of a reference spectrum I′_{ref} exhibiting a distinct peak P superimposed on a smooth baseline b(λ) is shown in FIG. 5.

As outlined in the introduction of the present disclosure, the negative impact of contributions of interfering influences on Raman spectroscopic measurements can be significantly reduced or even eliminated by determining and providing Raman difference spectra ΔI by subtracting correctly scaled reference spectra s₀*I_{ref} from the measured spectra Iₘ; e.g., by ΔI:=Iₘ−s₀*I_{ref}, wherein s₀ is a coefficient value of a scaling coefficient s.

Eliminating the contributions of the interfering influences included in the respective measured spectrum Iₘ by spectral subtraction requires for the coefficient value s₀ of the scaling coefficient s to be determined such that it corresponds to the magnitude of the contributions of the interfering influences included in the respective measured spectrum Iₘ. Under-subtracting by employing a first coefficient value s₁ of the scaling coefficient s that is too small, as well as over-subtracting by employing a second coefficient value s₂ of the scaling coefficient s that is too large, will modify the contributions of interfering influences included in the resulting Raman difference spectrum ΔI, but neither will not remove them.

The methods of the present disclosure recognize that the effects of contributions of interfering influences on the measured spectra Iₘ depend on the spectral shape of the reference spectrum I_{ref} and that the effects of peaks Pr1, Pr2, Pr3 that may be included in the reference spectrum I_{ref} have to be properly accounted for in the determination of coefficient value so.

In this respect, the method 200 includes a method step A3 of determining a difference spectrum function G corresponding to a difference between the measured spectrum Iₘ and a product of the scaling coefficient s and the reference spectrum I_{ref}.

In certain embodiments, the method 200 further includes a method step F of filtering the measured spectrum Iₘ and the reference spectrum I_{ref}. In such embodiments, the difference spectrum function G is determined based on the filtered measured spectrum FIₘ and the filtered reference spectrum FI_{ref}, e.g., by subtracting the product of the scaling coefficient s and the filtered reference spectrum FI_{ref} from the filtered measured spectrum FIₘ, e.g., by G:=FIₘ−s*FI_{ref}.

In method step F the filtering is, e.g., performed by with a smoothing filter filtering the measured spectrum Iₘ and the reference spectrum I_{ref}. In certain embodiments, the smoothing filter is, e.g., a second or higher order smoothing filter, or a Savitzky-Golay filter, e.g., a second or higher order Savitzky-Golay filter, e.g., a Savitzky-Golay filter having a window width of 20 to 30, e.g., a window width of 25, and a polynomial order of 3 or higher. As an alternative another type of smoothing filter, e.g., a wavelet decomposition filter, may be used instead.

The filtering provides the advantage of eliminating noise included in the measured spectrum Iₘ and the reference spectrum I_{ref} which may otherwise impair the determination of the coefficient value s₀, e.g., due to noise induced errors of the difference spectrum function G.

At the same time, the filtering provides the advantage of maintaining the spectral shape of the respective spectrum. Thus, the spectral intensity profile of the filtered measured spectrum FIₘ is at least approximately identical to the spectral intensity profile Iₘ(λ) of the unfiltered measured spectrum Iₘ, and the spectral intensity profile of the filtered reference spectrum FI_{ref} is at least approximately identical to the spectral intensity profile I_{ref}(λ) of the unfiltered reference spectrum I_{ref}.

In the method 200 disclosed herein, the determination of the coefficient value s₀ of the scaling coefficient s corresponding to the magnitude of the contributions of the interfering influences included in the respective measured spectrum Iₘ includes a method step A4 of determining a second derivative SDG of the difference spectrum function G with respect to a spectral line λᵢ and includes a method step A5 of determining the coefficient value so of the scaling coefficient corresponding to a magnitude of the contributions of the interfering influences included in the measured spectrum Iₘ such that the coefficient value so minimizes an error function E(s) representing an error of the difference spectrum function G as a function of the scaling coefficient S.

In method step A4, the second derivative SDG of the difference spectrum function G is, e.g., determined by subtracting the product of the scaling coefficient s and the second derivative d²/dλ² (FI_{ref}) of the filtered reference spectrum FI_{ref} from the second derivative d²/dλ² (FIₘ) of the filtered measured spectrum FIₘ, e.g., by SDG:=d²/dλ²(FIₘ)−s*d²/dλ²(FI_{ref}).

In embodiments, wherein a Savitsky-Golay filter is employed to filter the measured spectrum Iₘ and the reference spectrum I_{ref}, the filtering performed in method step F and the determination of the second derivative SDG performed in method step A4 is, e.g., performed in a combined single method step of performing a second order Savitsky-Golay differentiation of the difference spectrum function G here determined by subtracting product of the scaling coefficient s and the unfiltered reference spectrum I_{ref} from the unfiltered measured spectrum Iₘ, e.g., by Δ:=Iₘ−s*I_{ref}.

In method step A5, the effects of peaks Pr1, Pr2, Pr3 included in the reference spectrum I_{ref} are accounted for by the error function E(s) including a first term T1 corresponding to an error of the difference spectrum function G due to peaks Pr1, Pr2, Pr3 included in the reference spectrum I_{ref}.

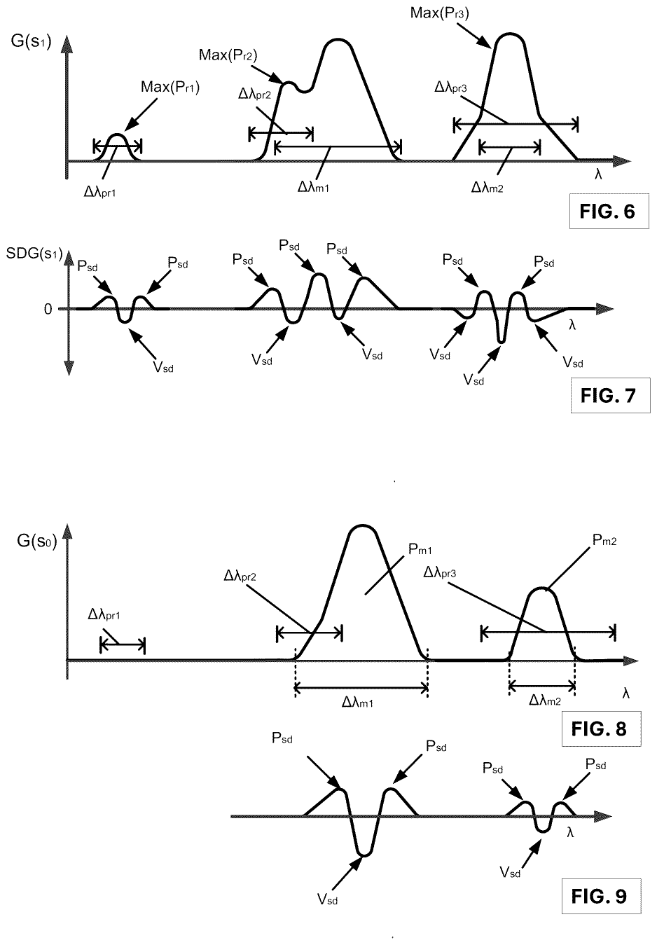

The nature of this error and its dependency on the value of the scaling coefficient s is subsequently described based FIGS. 6-11, which illustrate the principle shape of exemplary difference spectrum functions G(s₁), G(s₀), G(s₂) and the second derivatives G(s₁), G(s₀), G(s₂) thereof, which have been determined based on the measured spectrum Iₘ shown in FIG. 3 and the reference spectrum I_{ref} shown in FIG. 4.

In the intensity profile Iₘ(λ) of the measured spectrum Iₘ shown in FIG. 3, the magnitude and the spectral shape of the contributions of the interfering influences included in the measured spectrum Iₘ are indicated as shaded areas. Each shaded area corresponds to one of the peaks Pr1, Pr2, Pr3 of the reference spectrum I_{ref}. The exemplary peaks Pr1, Pr2, Pr3 of the reference spectrum I_{ref} shown in FIG. 4 occur in different positions with respect to other spectral features Pm1, Pm2, e.g., peaks, included in the measured spectrum Iₘ.

As shown in FIG. 3, the first peak Pr1 of the reference spectrum I_{ref} occurs in a first spectral region Δλpr1, wherein the measured spectrum Iₘ does not include any other spectral features. This first peak Pr1 causes the measured spectrum Iₘ to include an additional first maximum Max(Pr1) in the first spectral region Δλpr1. The second peak Pr2 of the reference spectrum I_{ref} occurs in a second spectral region Δλpr2, which partially overlaps with a spectral region Δλₘ₁, wherein the measured spectrum Iₘ includes the first spectral feature Pm1. Correspondingly the second peak Pr2 causes the measured spectrum Iₘ to include an additional second maximum Max(Pr2) superimposed on one side of the first spectral feature Pm1. The third peak Pr3 of the reference spectrum I_{ref} occurs in a third spectral region Δλpr3 covering a spectral region Δλm2, wherein the measured spectrum Iₘ includes the second spectral feature Pm2. In the example shown, the third peak Pr3 causes the measured spectrum Iₘ to include a third maximum Max(Pr3) exhibiting a maximum intensity exceeding the peak intensity of the second spectral feature Pm2 and a peak width exceeding the peak width of the second spectral feature Pm2 in the third spectral region Δλpr3.

FIG. 6 shows the difference spectrum function G(s₁) at a first coefficient value s₁ that is smaller than the coefficient value so corresponding to the magnitude of the interfering influences included in the measured spectrum Iₘ. FIG. 7 shows the second derivative SDG(s₁) of the difference spectrum function G(s₁) at this first coefficient value s₁. The first coefficient value s₁ being too small results in under-subtraction.

FIG. 8 shows the difference spectrum function G(s₀) at the coefficient value so corresponding to the magnitude of the interfering influences included in the measured spectrum Iₘ. FIG. 9 shows the second derivative SDG(s₀) of the difference spectrum function G(s₀) at this coefficient value s₀.

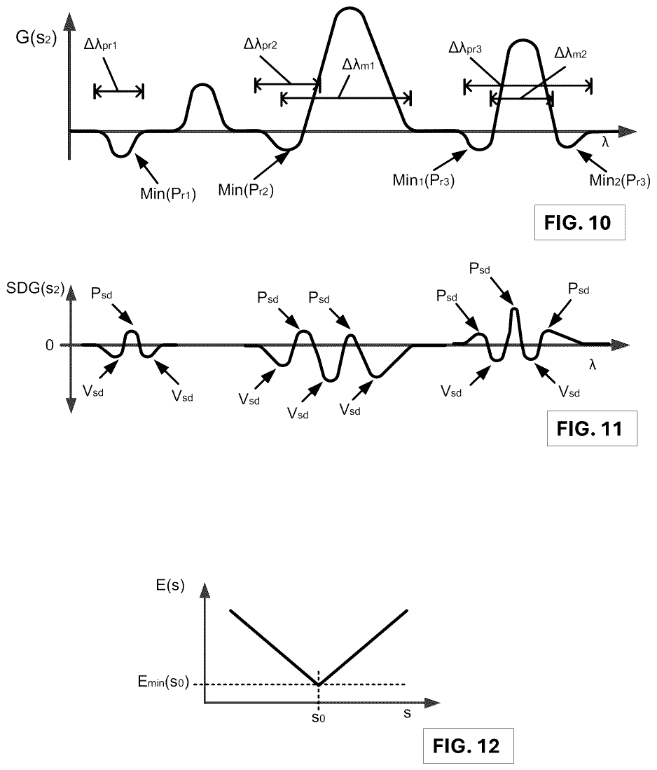

FIG. 10 shows the difference spectrum function G(s₂) at a second coefficient value s₂ that is larger than the coefficient value s₀ corresponding to the magnitude of the interfering influences included in the measured spectrum Iₘ. FIG. 11 shows the second derivative SDG(s₂) of the difference spectrum function G(s₂) at this second coefficient value s₂. The second coefficient value s₂ being too large results in over-subtraction.

As shown in FIGS. 6 and 7, under-subtraction of the first peak Pr1 of the reference spectrum I_{ref} reduces the pronouncedness of the first maximum Max(Pr1) but fails to eliminate the first maximum Max(Pr1). This causes the second derivative SDG(s₁) shown in FIG. 7 two exhibit a valley Vsd surrounded by two peaks Psd corresponding to the points of inflection the difference spectrum function G(s₁) shown in FIG. 6 in the first spectral region Δλpr1.

As shown in FIGS. 8 and 9, applying the scaling coefficient value s corresponding to the magnitude of the interfering influences included in the measured spectrum Iₘ eliminates the contributions of the first peak Pr1 of the reference spectrum I_{ref} in the difference spectrum function G(s₀). Correspondingly the second derivative SDG(s₀) does not exhibit any peaks and the valleys in the first spectral region Δλpr1.

Over-subtracting the first peak Pr1 of the reference spectrum I_{ref} causes the difference spectrum function G(s₂) shown in FIG. 10 to exhibit a minimum Min(Pr1). This causes the second derivative SDG(s₂) shown in FIG. 11 two exhibit a peak Psd surrounded by two valleys Vsd in the first spectral region Δλpr1 corresponding to the points of inflection of the difference spectrum function G(s₂) shown in FIG. 10.

In an analogous manner, under-subtracting the partially overlapping second peak Pr2 of the reference spectrum I_{ref} reduces the pronouncedness of the second maximum Max(Pr2) shown in FIG. 6, and over-subtracting the second peak Pr2 causes the difference spectrum function G(s₂) shown in FIG. 10 to exhibit a minimum Min(Pr2). As is apparent from a comparison of second derivatives SDG(s₁), SDG(s₀), SDG(s₂), shown in FIGS. 7, 9 and 11, respectively, under-subtraction and over-subtraction of the second peak Pr2 of the reference spectrum I_{ref} causes the second derivative Gsd(s₁), Gsd(s₂) to exhibit additional peaks Psd and valleys Vsd that are eliminated, as shown in FIG. 9, when the coefficient value s₀ corresponding to the magnitude of the interfering influences included in the measured spectrum Iₘ is applied.

With respect to the entirely overlapping third peak Pr3 of the reference spectrum I_{ref} under-subtracting the third peak Pr3 as shown in FIG. 6 and over-subtracting the third peak Pr3 as shown in FIG. 10 both affect the peaky spectral shape of the respective difference spectrum function G(s₁), G(s₂) in the third spectral range Δλpr3 in a manner causing the corresponding second derivatives SDG(s₁), SDG(s₂) shown in FIGS. 7 and 11 to exhibit additional peaks Psd and valleys Vsd, that are eliminated as shown in FIG. 9, when the coefficient value so corresponding to the magnitude of the interfering influences included in the measured spectrum Iₘ is applied.

As is apparent from this example, over-subtracting and under-subtracting peaky reference spectra I_{ref} causes the second derivative SDG to exhibit additional peaks Psd and valleys Vsd. These additional peaks Psd and valleys Vsd occur regardless of the position of peaks Pr1, Pr2, Pr3 included in the reference spectrum I_{ref} with respect to other spectral features Pm1, Pm2 included in the measured spectrum Iₘ.

Taking the first coefficient value s₁ as a starting point, gradually increasing the value of the scaling coefficient s up to the correct coefficient value so reduces the pronouncedness of the additional peaks Psd included in the second derivative SDG due to the incorrect size of the scaling coefficient s such that they are eliminated when the scaling coefficient s reaches the correct coefficient value so. Further increasing the scaling coefficient s to values exceeding the correct coefficient value so causes the additional peaks Psd included in the second derivative SDG due to under-subtraction to turn into valleys Vsd exhibited by second derivative SDG due to over-subtraction. Analogously, taking the first coefficient value s₁ as a starting point, gradually increasing the value of the scaling coefficient s up to the correct coefficient value so reduces the pronouncedness of the additional valleys Vsd included in the second derivative SDG due to the incorrect size of the scaling coefficient s such that they are eliminated when the scaling coefficient s reaches the correct coefficient value so. Further increasing the scaling coefficient s to values exceeding the correct coefficient value so causes the additional valleys Vsd included in the second derivative SDG due to under-subtraction to turn into peaks Psd exhibited by the second derivative SDG due to over-subtraction.

This effect enables for the error of the difference spectrum function G due to peaks Pr1, Pr2, Pr3 included in the reference spectrum I_{ref} to be quantitatively determined as a function of the scaling coefficient s based on the size of the areas enclosed underneath the second derivative SDG, in the spectral regions where the second derivative SDG exhibits peaks Psd and/or based on the size of the areas enclosed underneath the second derivative SDG, in the spectral regions where the second derivative SDG exhibits valleys Vsd.

Correspondingly, the first term T1 of the error function E(s) includes a first sum S1 given by the sum of areas enclosed underneath the second derivative SDG of the difference spectrum function G in all spectral regions in which the second derivative SDG exhibits peaks Psd. These spectral regions are given by the spectral regions in which the second derivative SDG of the difference spectrum function G is positive.

In addition or as an alternative, the first term T1 of the error function E(s) includes a second sum S2 given by the sum of areas enclosed underneath the second derivative SDG of the difference spectrum function G in all spectral regions in which the second derivative SDG exhibits valleys Vsd. These spectral regions are given by the spectral regions in which the second derivative SDG of the difference spectrum function G is negative.

In certain embodiments, the first sum S1 is, e.g., determined as an integral, e.g., a trapezoidal integral trapz, of the rectified linear unit relU of the spectral values SDG; of the second derivative SDG over the spectral range Δλ, e.g., by S1: =trapz (relU(SDGᵢ)), wherein the rectified linear unit relU (e.g., ramp function) of the spectral values SDGᵢ is equal to the absolute value of the spectral values SDGᵢ for all spectral values SDGᵢ having a positive value larger or equal to zero and is equal to zero for all spectral values SDGᵢ having a negative value.

Analogously the second sum S2 is, e.g., determined as an integral, e.g., a trapezoidal integral trapz of the rectified linear unit relU of minus one (−1) times the spectral values SDGᵢ of the second derivative SDG over the spectral range Δλ, e.g., by S2:=trapz (relU(−SDGᵢ)).

In certain embodiments, the first term T1 is, e.g., determined as term T1a given by a sum of the first sum S1 and the second sum S2. In this case, the first term T1 is, e.g., determined as an integral, e.g., a trapezoidal integral trapz, of the absolute values abs(SDGᵢ) of the spectral values SDGᵢ of the second derivative SDG over the spectral range Δλ, e.g., by:

T1a:=trapz(abs(SDGᵢ))=S1+S2

As an alternative, in certain embodiments, the first term T1 is, e.g., determined as term given by a weighted sum of the first sum S1 and the second sum S2.

As an example, in these embodiment, the first term T1 is, e.g., determined as a term T1b given by the sum of the first sum S1 and the product of a penalty factor pen₁ and the second sum S2, e.g., by:

T1b:=trapz(relU(Gsd,i))+pen₁*trapz(relU(−Gsd,i)).

As another example the first term T1 is, e.g., determined as a term T1c given by the sum of two products, wherein the first product is determined by multiplying the first sum S1 with a penalty factor pen₂, and the second product is determined by multiplying the difference between one and the penalty factor pen₂ with the second sum S2, e.g., by:

T1c:=pen₂*trapz(relU(Gsd,i))+(1−pen₂)*trapz(relU(−Gsd,i))

In applications in which the reference spectrum I_{ref} is a peaky spectrum, the spectral values SDGᵢ of the additional peaks Psd and valleys Vsd of the second derivative SDG caused by over-subtraction or under-subtraction of the peaks Pr1, Pr2, Pr3 included in the reference spectrum I_{ref} scale with size of the difference between scaling coefficient s and the coefficient value so corresponding to the magnitude of the contributions of the interfering influences included in the measured spectrum Iₘ. This causes the first term T1 to be a V-shaped function of the scaling coefficient s exhibiting a distinct minimum Eₘᵢₙ(s₀) at the coefficient value so corresponding to the magnitude of the contributions of the interfering influences included in the measured spectrum Iₘ. The V-shape provides the advantage of enabling an accurate determination of the coefficient value s₀ minimizing the first term T1.

Thus, in certain embodiments, the error function E(s) is, e.g., given by the first term T1, e.g., by E(s):=T1. This is illustrated in FIG. 12 showing an exemplary embodiment of the error function E(s) determined based on the measured spectrum Iₘ shown in FIG. 3 and the reference spectrum I_{ref} shown in FIG. 4, wherein the error function E(s) is given by the first term T1.

As an alternative, in certain embodiments, the error function E(s) employed in method step A5, e.g., includes at least one additional term. In these embodiments, the error function E(s) is, e.g., given by a sum or a weighted sum of the first term T1 and each additional term.

As an example, in certain embodiments, the error function E(s), e.g., includes a second term T2 corresponding to an error of the difference spectrum function G due to negative spectral intensity values Gi of the difference spectrum function G caused by over-subtraction.

Such an example is illustrated in FIG. 10 where over-subtraction of each of the three peaks Pr1, Pr2, Pr3 causes the spectral intensity values Gᵢ of the difference spectrum function G to be negative in the spectral regions in which the spectral intensity values of the product of the scaling coefficient s₂ and the reference spectrum I_{ref} is larger than the corresponding spectral intensity values I_{m,i} of the measured spectrum Iₘ. In FIG. 10, these regions are the spectral regions in which the difference spectrum function G exhibits the minima Min(Pr1), Min(Pr2), Min₁(Pr3), Min₂(Pr3).

The same effect of over-subtraction causing the difference spectrum function G to exhibit negative spectral intensity values Gᵢ in the spectral regions in which the spectral intensity values of the product of the scaling coefficient s and the reference spectrum I_{ref} are larger than the corresponding spectral intensity values I_{m,i} of the measured spectrum Iₘ also occurs in applications in which the reference spectrum I_{ref} is a smooth spectrum.

Regardless of whether the reference spectrum I_{ref} is peaky or smooth, the second term T2 is, e.g., determined as the sum of areas enclosed underneath the difference spectrum function G in all spectral regions in which the spectral intensity values Gi of the difference spectrum function G are negative. In certain embodiments, the second term T2 is, e.g., determined as an integral, e.g., a trapezoidal integral trapz of the rectified linear unit relU of minus one times the spectral intensity values Gᵢ of the difference spectrum function G over the spectral range, e.g., by T2:=trapz (relU(−Gsd,i)).

In certain embodiments, the error function E(s) is, e.g., given by the sum of the first term T1 and the product of a penalty factor pen and the second term T2, e.g., by E(s):=T1+pen*T2.

The E(s) including the second term T2 is especially advantageous in applications in which even an, in principle, negligibly small amount of over-subtraction is to be avoided, e.g., because further data processing of the Raman difference spectra ΔI determined based on the coefficient value s₀ is unable to cope with negative spectral intensity values of the Raman difference spectra ΔI, even if they are negligibly small.

Considering that the second derivative SDG of the difference spectrum function G is zero in all spectral regions in which the difference spectrum function G is constant or a linear function of the spectral line 2 and that the second term T2 on its own does not prevent under-subtraction, embodiments in which the error function E(s) given by the first term T1, as well as embodiments in which the error function E(s) given by a sum or the weighted sum of the first term T1 and the second term T2, e.g., by E(s):=T1+pen*T2, are preferably only employed in applications in which the reference spectrum I_{ref} is a peaky spectrum.

This can be ensured in various ways. In certain embodiments, this is, e.g., accomplished by limiting the applications where the method 200 disclosed herein is employed to applications in which the reference spectrum I_{ref} is a peaky spectrum.

In other embodiments, the method 200, e.g., includes a method step K of, in each application, differentiating whether the reference spectrum I_{ref} is a peaky spectrum or a smooth spectrum.

Based on this differentiation, the coefficient value so of the scaling coefficient s is then, e.g., only determined based on the error function E(s) given by the first term T1 or on the error function E(s) given by the sum or the weighted sum of the first term T1 and the second term T2, in case the reference spectrum I_{ref} is a peaky spectrum.

As an option, such an embodiment of the method 200, e.g., includes a further method step A5′ of determining the coefficient value s₀ such that it minimizes the replacement error function RE(s), in case the reference spectrum I_{ref} is a smooth spectrum.

The replacement error function RE(s) is, e.g., configured such that it exhibits a distinct minimum at the coefficient value so corresponding to the magnitude of the interfering influences included in the measured spectrum in applications in which the reference spectrum I_{ref} is a smooth spectrum.

With respect to the replacement error function RE(s), the functions described in the introduction of the present disclosure may be employed. As an example, a replacement error function RE(s) given by a variance of the second order derivative SDG of the difference spectrum function G may be employed. Another example is a replacement error function RE(s) given by a sum of a first term corresponding to an asymmetric weighted sum of the squares of the differences between the spectral intensity values I_{m,i} of the measured spectrum Iₘ and the scaled reference spectrum s*I_{ref} and a product of a penalty factor q and a second term corresponding to the sum of the squares of the second derivatives SDI_{ref,i} of the reference spectrum I_{ref} at individual spectral lines λᵢ, e.g., by:

$$RE ( s ) := {Σ}_{i = 1}^{n} {w}_{i} * {( {I}_{m , i} - s * {I}_{ref , i} )}^{2} + q * {{Σ}_{i = 1}^{n} ( {SDI}_{ref , i} )}^{2}$$

wherein the weighing factors wᵢ applied to the squares of differences due to spectral intensity values I_{m,i} of the measured spectrum I_{m,i} exceeding the corresponding spectral intensity values I_{ref,i} of the reference spectrum I_{ref} are substantially smaller than weighing factors wᵢ assigned to the squares of differences due to spectral intensity values I_{m,i} of the measured spectrum I_{m,i} subsiding the corresponding spectral intensity values I_{ref,i} of the reference spectrum I_{ref}.

With respect to the method step K of differentiating whether the reference spectrum I_{ref} is peaky or smooth, for some applications, it may be known in advance whether the reference spectrum I_{ref} is a peaky spectrum or not. In this case, the differentiation is, e.g., performed based on this prior knowledge.

As an alternative, in certain embodiments, the method step K, e.g., includes determining whether the reference spectrum I_{ref} is a peaky spectrum.

In such embodiments, the method step K, e.g., includes performing a visual inspection of the intensity profile I_{ref}(λ) of the reference spectrum I_{ref} and determining the reference spectrum I_{ref} to be a peaky spectrum in case it includes at least one distinct peak Pr1, Pr2, Pr3.

As an alternative, in certain embodiments, method step K, e.g., includes determining the baseline b(λ) of the reference spectrum I_{ref} and determining a residual spectrum R corresponding to a difference between the reference spectrum I_{ref} and the baseline b(λ), e.g., by R:=I_{ref}−b(λ). Following this determination, an indicator KP corresponding to a degree of peakiness of the residual spectrum R is determined.

In certain embodiments, the indicator KP is, e.g., determined based on the sum of the squares of the spectral intensity values Ri of the residual spectrum R, e.g., as a ratio of the sum of the squares of the spectral intensity values Ri divided by a mean RA of the spectral intensity values Ri, e.g., by:

$$KP := \frac{1}{RA} {Σ}_{i = 1}^{n} {R}_{i}^{2}$$

As an alternative, in certain embodiments, the indicator KP is, e.g., determined as or based on a variance of the residual spectrum or based on another function of the residual spectrum R corresponding to the degree of peakiness of the residual spectrum R.

Following the determination of the indicator KP, the reference spectrum I_{ref} is then determined to be a peaky spectrum in case the indicator KP is larger than a predetermined threshold Kr and/or determined to be a smooth spectrum in case the indicator KP is smaller than the threshold Kr.

Differentiating whether the reference spectrum I_{ref} is peaky or smooth, does however require at least some additional effort, e.g., due to additional computing power required and/or the increased complexity of the computer program executed by the difference spectra determination unit 120 performing the method 200 based on the measured spectra Iₘ and the reference spectrum I_{ref} provided to the difference spectra determination unit 120.

This burden can be avoided by determining the error function E(s) employed in method step A5 such that it can be used in applications in which the reference spectrum I_{ref} is a peaky spectrum, as well as in applications in which the reference spectrum I_{ref} is a smooth spectrum.

In certain embodiments, this is, e.g., achieved by the error function E(s) including the first term T1 and an additional function f(s) of the scaling coefficient s exhibiting a distinct minimum at the coefficient value s₀ corresponding to the magnitude of the interfering influences included in the measured spectrum Iₘ in applications in which the reference spectrum I_{ref} is a smooth spectrum. In such embodiments, the error function E(s) is, e.g., determined as a sum or a weighted sum of the first term T1 and the additional function f(s).

In certain embodiments, the error function E(s) including the first term T1 and the function f(s) is, e.g., given by a function including the first term T1 and the second term T2 described above and an additional third term T3 corresponding to an error of the difference spectrum function G due to under-subtraction. The third term T3 is, e.g., given by the sum of areas enclosed underneath the difference spectrum function G in all spectral regions in which the spectral intensity values Gi of the difference spectrum function G are positive. In this respect, the third term T3 is, e.g., determined as an integral, e.g., a trapezoidal integral trapz, of the rectified linear unit relU of the spectral intensity values Gᵢ of the difference spectrum function G over the spectral range Δλ, e.g., by T3:=trapz (relU(Gᵢ)).

In such embodiments, the error function E(s) is, e.g., determined as a sum or a weighted sum of the first term T1, the second term T2 and the third term T3, e.g., by E(s):=T1+pen₃*T2+T3, wherein pen₃ is a penalty factor applied to the second term T2 that penalizes over-subtraction.

The error function E(s) including the first term T1 and the function f(s), e.g., the function f(s) given by f(s):=pen₃*T2+T3, provides the advantage of being employable in method step A5 in each application, regardless of whether the reference spectrum I_{ref} is a peaky spectrum or a smooth spectrum. This provides the advantage that, in each application, it neither has to be known nor determined whether the reference spectrum I_{ref} is a peaky spectrum or not.

Using the error function E(s) including the first term T1 and the function f(s) in each application provides the advantage of simplifying the computer program installed on the difference spectra determination unit 120 (shown in FIG. 2) performing the determination of the coefficient value s₀. The function f(s) including the second term T2 and the third term T3 provides the advantage of needing very little computing power to determine both additional terms T2, T3. The error function E(s) including the function f(s) further provides the advantage that no replacement error function RE(s) is needed to cover applications in which the reference spectrum I_{ref} is not peaky, and that the method 200 shown in FIG. 1 can be or is performed without discriminating between peaky and smooth reference spectra I_{ref} and correspondingly also without performing the method step K of determining whether the reference spectra I_{ref} are peaky spectra.

Regardless of whether the error function E(s) employed in method step A5 only includes the first term T1, the first term T1 and the second term T2, or the first term T1 and the function f(s), the coefficient value so of the scaling coefficient s is determined such that it is equal to the value Min[E(s)] of the scaling coefficient s at which the error function E(s) exhibits its minimum Eₘᵢₙ(s).

In certain embodiments, in method step A5, the coefficient value so minimizing the error function E(s) is, e.g., determined by performing a golden section search, a particle swarm optimization method, or a gradient descent method. As an alternative, another method of determining the minimum of a function may be employed.

Following the determination of the coefficient value so, the method 200 further includes a method step A6 of, for at least one measured spectrum Iₘ determined by the Raman spectrometer 110, determining and providing a Raman difference spectrum ΔI corresponding to the difference between the respective measured spectrum Iₘ and the product of the coefficient value so and the reference spectrum I_{ref}.

In certain embodiments, the method 200, e.g., includes with the Raman spectrometer 110 repeatedly determining and providing measured spectra Iₘ and, for each measured spectrum Iₘ, determining and providing the corresponding Raman difference spectrum ΔI.

In this case, for each measured spectrum Iₘ the Raman difference spectrum ΔI is, e.g., determined by, based on the respective measured spectrum Iₘ, determining the coefficient value s₀ as described above and, based on the thus determined coefficient value so, determining the Raman difference spectrum ΔI. This is particularly advantageous in applications in which the magnitude of the contributions of the interfering influences included in the measured spectra Iₘ may change rapidly.

As an alternative, in certain embodiments, the coefficient value so determined based on a previously determined measured spectrum Iₘ is, e.g., employed to determine Raman difference spectra ΔI of a series of subsequently determined measured spectra Iₘ. In such embodiments, the coefficient value s₀ is, e.g., employed for all subsequently determined measured spectra Iₘ. As an alternative, the coefficient value so is, e.g., updated at least once, repeatedly or periodically, by repeating the previously performed method of determining the coefficient value so based on a newly determined measured spectrum Iₘ. The latter is particularly advantageous in applications in which the magnitude of the contributions of the interfering influences included in the measured spectra Iₘ may change on a time scale that is long compared to the time intervals between consecutively determined measured spectra Iₘ.

Regardless of how frequently the coefficient value so is determined and/or updated, the Raman difference spectra ΔI determined and provided in method step A6 may be used in various way.

As an example, in certain embodiments, the method 200 disclosed herein, e.g., includes a method step A7 of, based on at least one or each Raman difference spectrum ΔI determined in method step A6, determining a measurement result MR of at least one measurand of the medium. In this respect, the Raman spectroscopic measurement system 100 shown in FIG. 2, e.g., includes a signal processing unit 130, e.g., a computer, a microprocessor or another type of calculating unit, determining and providing the measurement results MR based on the Raman difference spectra ΔI provided by the difference spectra determination unit 120 and on a previously determined model MOD for determining the measurement results MR based on spectral intensity values of the Raman difference spectra ΔI.

In addition or as an alternative, in certain embodiments, the method 200, e.g., includes a method step A8 of, for at least one measurand based on Raman difference spectra ΔI determined as described above for measured spectra Iₘ of samples SM of media exhibiting known values of the respective measurand, determining the model MOD for determining measurement results MR of the respective measurand. Determining the model MOD, e.g., includes, based on the Raman difference spectra ΔI and the corresponding known values of the respective measurand, identifying spectral features associated to the measurand and/or interrelations between spectral features associated to the measurand and determining and providing an algorithm for calculating measurement results MR of the measurand based on selected spectral intensity values of the Raman difference spectra ΔI. In certain embodiments, at least one thus determined model MOD is, e.g., subsequently used in method step A7 to determine the measurement result(s) MR.

Determining measurement results MR and/or determining at least one model MOD for determining measurement results MR based on the Raman difference spectra ΔI provides the advantage that spectral information on the measurands included in the Raman difference spectra ΔI is no longer obscured by the contributions of the interfering influences included in the measured spectra Iₘ. Thus, accurate models MOD and/or a high measurement accuracy of the measurement results MR is achieved.

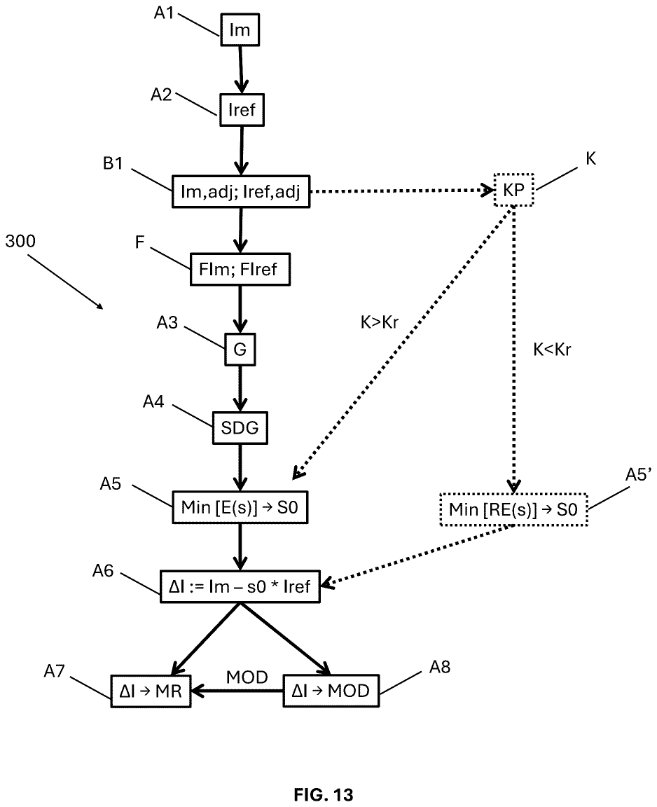

In a further aspect of the present disclosure, an embodiment of a method 300 is shown in FIG. 13. The method 300 includes method step A1, in which the Raman spectrometer acquires at least one measured spectrum Iₘ of the medium, and method step A2, in which at least one spectrum I_{ref} containing the contributions of the interfering influences included in the measured spectrum Iₘ is provided. Method steps A1 and A2 of the method 300 are performed as described with respect to the steps A1 and A2 of the method 200.

In at least one embodiment, the method 300 includes method step B1, wherein the measured spectrum Iₘ and the reference spectrum I_{ref} are preprocessed to produce at least one adjusted measuring spectrum I_{m,adj} and an adjusted reference spectrum I_{ref,adj}. The preprocessing step B1 advantageously improves the suitability of the spectra for subsequent method steps, ultimately improving the quality of the measurement results.

In some embodiments of the method 300, the preprocessing step B1 includes trimming the measured spectra Iₘ and/or the reference spectra I_{ref} to exclude certain wavelength regions from subsequent method steps. For example, non-informative regions of the spectra, or those that are prone to interfering influences, such as instrument noise or Rayleigh scattering, can be removed. Such trimming of the spectra leads, for example, to improvements in the signal-to-noise ratio, thus improving the fit quality of subsequent method steps. Further, when the measurement medium or analyte is an organic compound, such as an alkane, the preprocessing step B1 may include trimming the measured spectra Iₘ and/or the reference spectra I_{ref} to include only the spectral region defined by 250-3245 cm⁻¹.

In some embodiments of the method 300, the preprocessing step B1 and/or the method step F of filtering the measured spectrum Iₘ and the reference spectrum I_{ref} includes applying a background reduction or detection procedure. Employing a background reduction or detection procedure advantageously helps isolate true signal from spectral noise, further improving the quality of the analysis. For example, in some embodiments, the preprocessing step B1 includes applying a rolling ball filter or procedure to the measured spectra Iₘ and/or the reference spectra I_{ref}. The rolling ball filter or procedure discerns slowly evolving background features or broad spectral noise by simulating a ball rolling beneath the spectral surface. As the ball cannot traverse sharp peaks or narrow features, its path defines a smooth baseline. Subtracting this baseline from the spectrum helps eliminate background noise, thereby enhancing the clarity of significant spectral details.

In some embodiments of the method 300, the preprocessing step B1 includes applying standard normal variate (“SNV”) normalization to the measured spectra Iₘ and to the reference spectra I_{ref}.

SNV normalization includes steps of subtracting the mean of a spectrum from each data point of the spectrum and dividing the result by the standard deviation. The SNV normalization produces spectra that have a mean equal to zero and a standard deviation equal to one. As an alternative to performing SNV normalization on the reference spectra I_{ref} at runtime, the reference spectra I_{ref} may be provided as previously SNV normalized, or with a mean equal to zero and a standard deviation equal to one.

SNV normalization enhances the quality and reliability of subsequent spectral analysis by improving comparability across samples and mitigating the effects of physical artifacts, such as baseline shifts and scattering variations.

Upon performing the preprocessing step B1 of method 300 on the measured spectra and the reference spectra, at least one adjusted measured spectra I_{m,adj} and at least one adjusted reference spectra I_{ref,adj} are obtained, respectively. The adjusted measured spectra I_{m,adj} and adjusted reference spectra I_{ref,adj} can then be used analogously to the measured spectra Iₘ and the reference spectra I_{ref} as described for the method 200. For example, the adjusted measured spectra I_{m,adj} and the adjusted reference spectra I_{ref,adj} may be used as inputs to the filtering step F to produce the filtered measured spectrum FIₘ and the filtered reference spectrum FI_{ref}. Further, in certain embodiments of the method 300, the preprocessing step B1 can be performed concurrently with the filtering step F, as described with respect to the method 200.

Accordingly, the method 300 allows a Raman difference spectrum ΔI to be determined from the adjusted measured spectra I_{m,adj} and the adjusted reference spectra I_{ref,adj} via method steps F and A3-A8, which can be applied as described for the method 200.

Specifically, the method 300 includes method step A3, wherein a difference spectrum function G is calculated as the difference between the adjusted measured spectrum I_{m,adj} and a product of a scaling coefficient s and the adjusted reference spectrum I_{ref,adj}.

In method step A4, the second derivative SDG of the difference spectrum function G is computed with respect to a spectral line λⱼ.

Method step A5 includes determining the coefficient value so of the scaling coefficient corresponding to a magnitude of the contributions of the interfering influences included in the adjusted measured spectrum I_{m,adj} such that the coefficient value so minimizes an error function E(s) representing an error of the difference spectrum function G as a function of the scaling coefficient s.

In method step A6, for at least one measured spectrum Iₘ, the Raman difference spectrum ΔI is computed as the difference between the respective measured spectrum Iₘ and the product of the coefficient value so and the reference spectrum I_{ref}.

In certain embodiments, the method 300 includes a method step A7 of, based on at least one or each Raman difference spectrum ΔI determined in method step A6, determining a measurement result MR of at least one measurand of the medium.

In certain embodiments, the method 300 includes method step A8, which may be used in addition to or as an alternative to method step A7. In method step A8, a model MOD is developed to determine measurement results MR based on Raman difference spectra ΔI, wherein the Raman difference spectra ΔI are derived as described above from measured spectra Iₘ of samples SM of media exhibiting known values of the respective measurand.

Further, in at least one embodiment of the method 300 according to the present disclosure, the model MOD in method step A8 includes a partial least squares (“PLS”) regression model. The PLS model may be trained by correlating Raman difference spectra ΔI of samples SM of media exhibiting known values of the measurand. The resulting PLS model defines a regression function that enables the calculation of measurement results MR from selected spectral intensity values of the Raman difference spectra ΔI. As an example, the PLS model MOD thus obtained may be subsequently applied in method step A7 to determine measurement results MR for unknown samples. For example, the model MOD may be employed to determine a pH-value of the medium, a melt index of the medium, a cell motility of the medium, and/or concentrations of components included in the medium, for example, the concentration of alkanes in an oil medium.

The method 300 advantageously provides that spectral contributions from interfering influences present are substantially reduced or eliminated. As a result, the spectral information relevant to the measurand is more clearly resolved, thereby enabling the development of more accurate and robust models and improving the precision and reliability of determined measurement results.

For example, Applicant has discovered that the method 300 can improve the detection limit by over 90% compared to conventional subtraction methods. Further, in the case of alkanes in oil, for example, pentane in canola oil, Applicant has found the detection limit of the method 300 to be less than 1,000 ppm.

The method 300 is also applicable to monitoring residual alkanes in other edible oils and edible oil byproducts post-extraction, such as residual alkanes in solid bean waste and/or bean meal. For example, after initial oil extraction from beans, for example soybeans, the method 300 can assess residual alkane levels of the resultant meal byproduct. These results can then advantageously inform decisions on whether additional processing steps are necessary.

Importantly, the method 300 of present disclosure is not limited to the measurement of alkanes or oil. Given the benefit of the present disclosure, a person of ordinary skill in the art would recognize that the method 300 may also be applied for measuring other mediums. This includes the detection of other hydrocarbon families and related compounds, including, but not limited to, alkenes, alkynes, cyclic hydrocarbons, and hydrocarbons with various heteroatoms and/or functional groups. The principles of the present disclosure offer benefits for measuring each of these compounds.

In a further aspect of the present disclosure, a Raman spectroscopic measurement system 100 capable of performing the method 300 of the present disclosure is shown in FIG. 2. The Raman spectroscopic measurement system 100 includes a Raman spectrometer 110 configured for determining and providing measured spectra Iₘ of the medium and a difference spectra determination unit 120 configured for determining and providing corresponding Raman difference spectra ΔI based on the measured spectra Iₘ.

The difference spectra determination unit 120 is, e.g., a computer, a microprocessor or another type of programmable calculating device including a memory, e.g., non-transitory computer readable medium, and configured to determine the Raman difference spectra ΔI based on the measured spectra Iₘ by executing a computer program, e.g., instructions executable by a processor, installed on the difference spectra determination unit 120. In accordance with the present disclosure, the difference spectra determination unit 120 is configured to perform the method 300.

While various embodiments of a method Raman spectrometer and methods for using and constructing the same have been described in considerable detail herein, the embodiments are merely offered by way of non-limiting examples of the disclosure described herein. It will therefore be understood that various changes and modifications may be made, and equivalents may be substituted for elements thereof, without departing from the scope of the disclosure. The present disclosure is not intended to be exhaustive or to limit the scope of the subject matter of the disclosure.

Further, in describing representative embodiments, the disclosure may have presented a method and/or process as a particular sequence of steps. However, to the extent that the method or process does not rely on the particular order of steps set forth herein, the method or process should not be limited to the particular sequence of steps described. Other sequences of steps may be possible and thus remain within the scope of the present disclosure.

1. A method of Raman spectroscopy for performing Raman spectroscopic measurements of at least one measurand of a medium in at least one application, wherein measured spectra of the medium include contributions of interfering influences that at least partially obscure spectra features associated to the at least one measurand, the method comprising, in at least one application:
with a Raman spectrometer, determining a measured spectrum of the medium;
providing a reference spectrum of the contributions of the interfering influences included in a respective measured spectrum, wherein a mean of the reference spectrum is equal to zero and a standard deviation of the reference spectrum is equal to one;
determining an adjusted spectrum from the measured spectrum of the medium, wherein a mean of the adjusted spectrum is equal to zero, and a standard deviation of the adjusted spectrum is equal to one;
determining a difference spectrum function corresponding to a difference between a product of a scaling coefficient and the reference spectrum;
determining a second derivative of the difference spectrum function with respect to a spectral line;
determining a coefficient value of the scaling coefficient corresponding to a magnitude of the contributions of the interfering influences included in the respective adjusted spectrum such that the coefficient value minimizes an error function representing an error of the difference spectrum function as a function of the scaling coefficient, wherein the error function includes a first term corresponding to an error exhibited by the difference spectrum function due to peaks included in the reference spectrum, the first term including at least one of:
a first sum of areas enclosed underneath the second derivative of the difference spectrum function in all spectral regions in which the second derivative of the difference spectrum function is positive; and
a second sum of areas enclosed underneath the second derivative of the difference spectrum function in all spectral regions in which the second derivative of the difference spectrum function is negative; and
determining and providing a Raman difference spectrum corresponding to the difference between the adjusted spectrum and a product of the coefficient value and the reference spectrum.

2. The method according to claim 1, further comprising determining a measurement result of the at least one measurand of the medium based on the Raman difference spectrum or based on more than one Raman difference spectrum.

3. The method according to claim 1, further comprising, for the at least one measurand, determining a model for determining measurement results of each respective measurand based on the Raman difference spectrum determined from adjusted spectra of samples of media exhibiting known values of the respective measurand.

4. The method according to claim 3, wherein the model for determining measurement results of the respective measurand comprises a partial least squares regression, wherein the partial least squares regression is fitted from Raman difference spectra of samples of media exhibiting known values of the respective measurand.

5. The method of claim 4, wherein the respective measurand comprises the concentration of an analyte in the medium.

6. The method of claim 5, wherein the medium comprises oil, and the analyte is an alkane in the oil.

7. The method of claim 6, wherein the concentration of the alkane in the medium is detected at a detection limit of less than 1,000 ppm.

8. The method of claim 5, wherein the medium comprises bean meal byproduct from an initial oil extraction, and the analyte is an alkane in the bean meal byproduct.

9. The method of claim 1, wherein the step of determining the adjusted measuring spectrum and adjusted reference spectrum comprises trimming the measured spectrum and/or the reference spectrum such that at least one portion of the measured spectrum and/or the reference spectrum is excluded from subsequent method steps.

10. The method according to claim 1, further comprising:
with a smoothing filter, a second or higher order smoothing filter, a Savitzky-Golay filter, a second or higher order Savitzky-Golay filter, a rolling ball filter, or a wavelet decomposition filter, filtering the adjusted spectrum and the reference spectrum; and
determining the difference spectrum function and/or the second derivative of the difference spectrum function based on the filtered adjusted spectrum and the filtered reference spectrum.

11. A Raman spectroscopic measurement system configured to perform the method according to claim 1, the Raman spectroscopic measurement system comprising:
a Raman spectrometer configured to determine and provide measured spectra of a medium; and
a difference spectra determination unit configured to determine and provide Raman difference spectra based on the measured spectra and a reference spectrum of contributions of interfering influences included in the measured spectra provided to the difference spectra determination unit.

12. A Raman spectroscopic measurement system, comprising:
a Raman spectrometer, comprising:
a monochromatic light source configured to transmit excitation light having a predetermined excitation wavelength a measurement region configured to accommodate a sample of a medium as to illuminate the sample; and
a spectrometric unit configured to receive Raman scattered light emanating from the illuminated sample and to provide measured spectra, including intensity spectra of the Raman scattered light, wherein the spectrometric unit includes a disperser, a detector, and a signal processor configured to determine and to provide spectral intensity values of the measured spectra based on signals from the detector; and
a difference spectra determination unit configured to determine Raman difference spectra based on the measured spectra
wherein the signal processor of the spectrometric unit and the difference spectra determination unit are cooperatively configured to perform the method according to claim 1.

13. The Raman spectroscopic measurement system of claim 12, further comprising a signal processing unit configured to determine and provide a measurement result of the at least one measurand of the medium based on the Raman difference spectrum provided by the difference spectra determination unit and on a previously determined model for determining the measurement result based on spectral intensity values of the Raman difference spectra.

14. The Raman spectroscopic measurement system of claim 13, wherein the previously determined model is a partial least squares regression model fitted from Raman difference spectra of samples of media exhibiting known values of the at least one measurand.

15. A non-transitory computer-readable medium storing instructions that, when executed by one or more programmable processors, cause the one or more programmable processors to perform operations for performing a method of Raman spectroscopy comprising determining and providing Raman difference spectra for performing Raman spectroscopic measurements of at least one measurand of a medium in at least one application, wherein measured spectra of the medium include contributions of interfering influences that at least partially obscure spectra features associated to the at least one measurand, based on a measured spectrum of the medium determined by a Raman spectrometer and a reference spectrum of the contributions of the interfering influences included in a respective measured spectrum, the operations comprising:
determining a second derivative with respect to a spectral line of a difference spectrum function corresponding to a difference between the respective measured spectrum and the product of a scaling coefficient and the reference spectrum;
determining a coefficient value of the scaling coefficient corresponding to a magnitude of the contributions of the interfering influences included in the respective measured spectrum such that the coefficient value minimizes an error function representing an error of the difference spectrum function as a function of the scaling coefficient, wherein the error function includes a first term corresponding to an error exhibited by the difference spectrum function due to peaks included in the reference spectrum, the first term including at least one of:
a first sum of areas enclosed underneath the second derivative of the difference spectrum function in all spectral regions in which the second derivative of the difference spectrum function is positive; and
a second sum of areas enclosed underneath the second derivative of the difference spectrum function in all spectral regions in which the second derivative of the difference spectrum function is negative;
for at least one measured spectrum determined by the Raman spectrometer, determining a Raman difference spectrum corresponding to the difference between the respective measured spectrum and the product of the coefficient value and the reference spectrum; and
applying a previously determined model to the Raman difference spectrum to determine a measurement result of the medium.

16. The non-transitory computer-readable medium of claim 15, wherein the previously determined model comprises a partial least squares regression.
